(19) United States
(12) Patent Application Publication
(10) Pub. No.: US 2007/0268993 A1
(43) Pub. Date: Nov. 22, 2007

(54) Computed Tomography Method and Computer Tomograph for Reconstruction of Object Images from Real and Fictitious Measured Values

(75) Inventors: Peter Forthmann, Hamburg (DE); Thomas Kohler, Norderstedt (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/573,714

(22) PCT Filed: Aug. 10, 2005
(86) PCT No.: PCT/IB05/52645

§ 371 Date: Feb. 15, 2007

(30) Foreign Application Priority Data
Aug. 20, 2004 (EP) 04104012.2

(51) Int. Cl. A61B 6/03

(52) U.S. Cl. 378/4

(57) ABSTRACT

The invention relates to a computed tomography method in which a radiation source moves relative to an object on a helical trajectory, where the movement comprises a rotation around an axis of rotation and a displacement parallel to the axis of rotation. During the movement real measured values are acquired by means of a detector unit. First a provisional object image is constructed from the real measured values, from which provisional object image fictitious, non-acquired measured values can be determined by deriving the provisional object image in a direction parallel to the axis of rotation. An image of the examination area is reconstructed from the real and fictitious measured values.

The invention relates to a computed tomography method, in which a radiation source generating a conical bundle of rays is moved relative to an object on a helical trajectory. The object is passed through by the bundle of rays and real measured values dependent on the intensity of the bundle of rays on the other side of the object are measured. Non-acquired, fictitious measured values are determined from the real measured values and an object image is reconstructed from the real and fictitious measured values. The invention further relates to a computer tomograph comprising a detector unit coupled to a radiation source for acquisition of real measured values, a computing unit for determining fictitious values from real measured values and for reconstructing an object image from the real measured values and the fictitious values. Furthermore, the invention relates to a computer tomograph for implementing the method and to a computer program for controlling the computer tomograph.

A computed tomography method of the type mentioned in the opening paragraph is published in, Improved two-dimensional rebinning of helical cone-beam computerized tomography data using John's equation”, M. Defrise, F. Noo and H. Kudo, Inverse Problems 19 (2003) S41-S54, Institute of Physics Publishing (further denoted by E1). F. Fictitious measured values are calculated in E1 from the real measured values with the help of the known John-equation. The calculation of the fictitious measured values is very complex and therefore susceptible to wrong calculations, which leads to artifacts in the reconstructed object image and thereby to a poor image quality.

It is therefore an object of the present invention to indicate a computed tomography method of the type mentioned in the opening paragraph and a computer tomograph, in which the quality of the reconstructed object image is improved in comparison with the method described in E1.

This object is achieved by a computed tomography method comprising the following steps:

a) Determination of non-acquired, fictitious measured values from the real measured values, where a fictitious beam is assigned to each fictitious measured value and where the determination of a fictitious measured value has the following steps:

i) Reconstruction of a provisional object image from the real measured values,

ii) Calculation of a partial derivation of the provisional object image in a direction parallel to the axis of rotation,

b) Reconstruction of a final object image from the real measured values and the fictitious measured values.

Furthermore, this object is achieved by a computer tomograph comprising a detector unit coupled to a radiation source for acquisition of real measured values, a computing unit for determining fictitious values from real measured values and for reconstructing an object image from the real measured values and the fictitious values.

In contrast to the computed tomography method described in E1, fictitious measured values are obtained not with the help of the John equation, but with the help of a partial derivation of the real measured values in a direction parallel to the axis of rotation. This partial derivation is obtained by reconstructing a provisional object image, deriving the provisional object image in the direction parallel to the axis of rotation and making a forward projection by means of the partially derived provisional object image along a real beam. These steps can be executed easily and without approximations, such that the reconstructed, final object image has an image quality improved in comparison to the current state-of-the-art technology published in E1.

The reconstruction of a provisional object image and/or the final object image with an exact reconstruction method as claimed in claim 3 leads to further improvement in the image quality.

The reconstruction of the provisional object image with lower resolution than the final object image as claimed in claim 4 leads to a reduction in the computation expenditure.

The embodiments as claimed in the claims 5 to 8 lead to a further improvement in the image quality of the reconstructed final object image.

Claim 10 defines a computer program for controlling a computer tomograph as claimed in claim 1.

The invention will be elucidated in detail below with the help of the drawings.

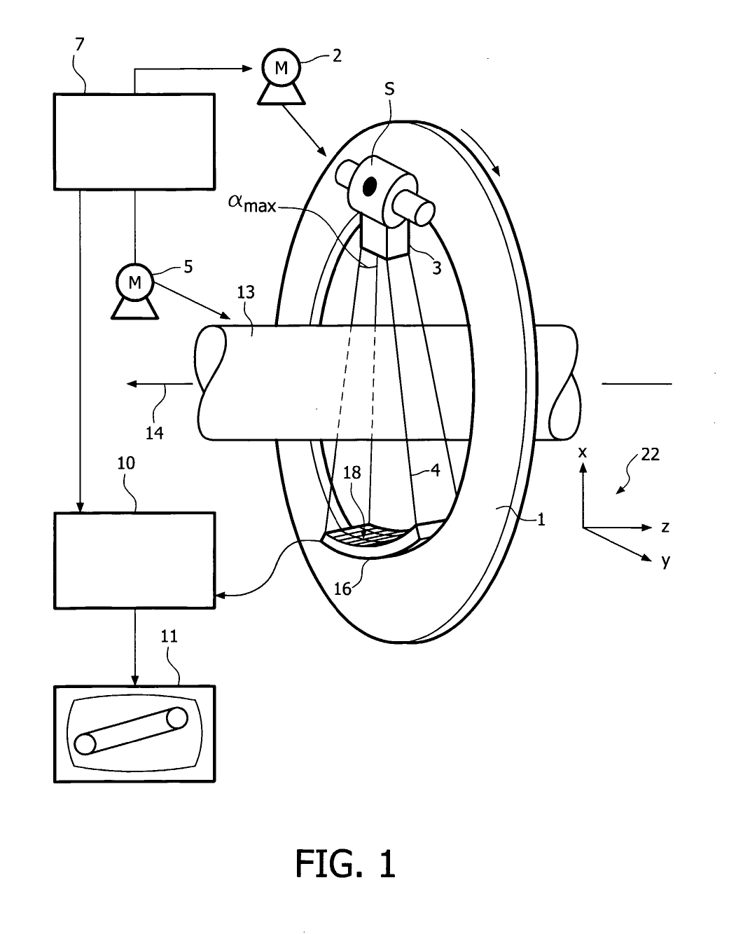

FIG. 1 A computer tomograph, by means of which the method as invented can be executed,

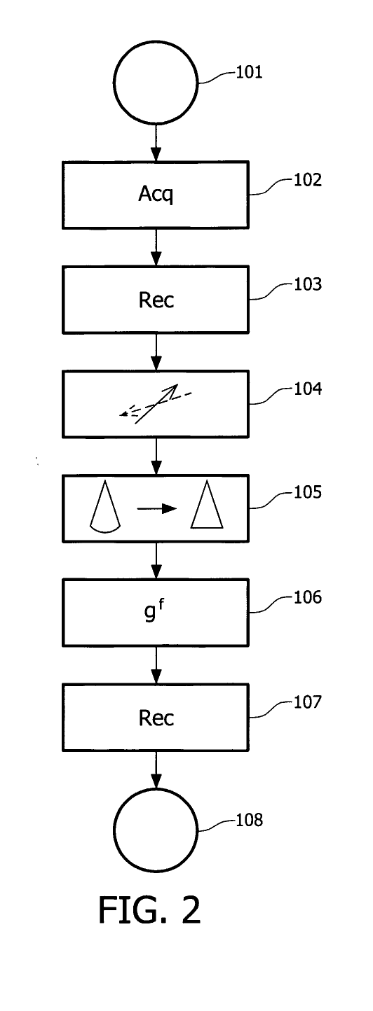

FIG. 2 A flow chart of an embodiment of the method according to the invention,

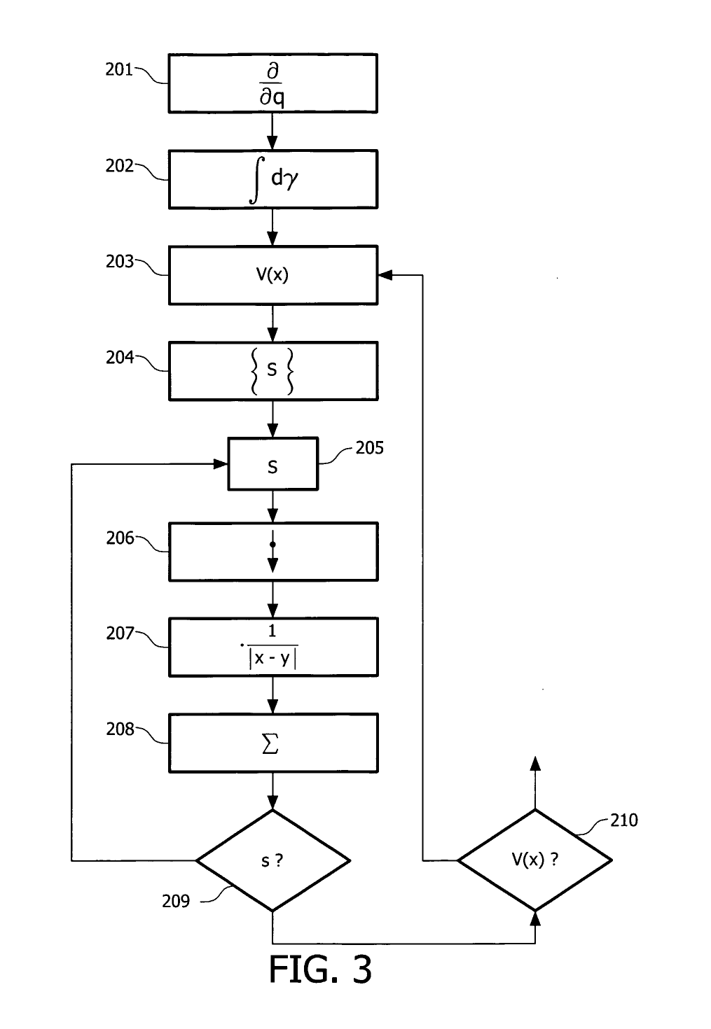

FIG. 3 A flow chart of an exact reconstruction by using a K method,

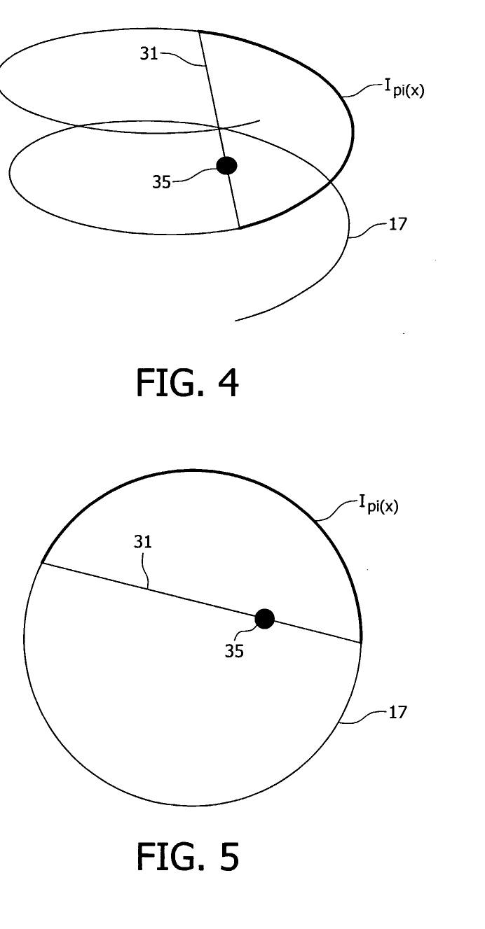

FIG. 4 A schematic perspective view of a helical section,

FIG. 5 A schematic plan view of the helical section,

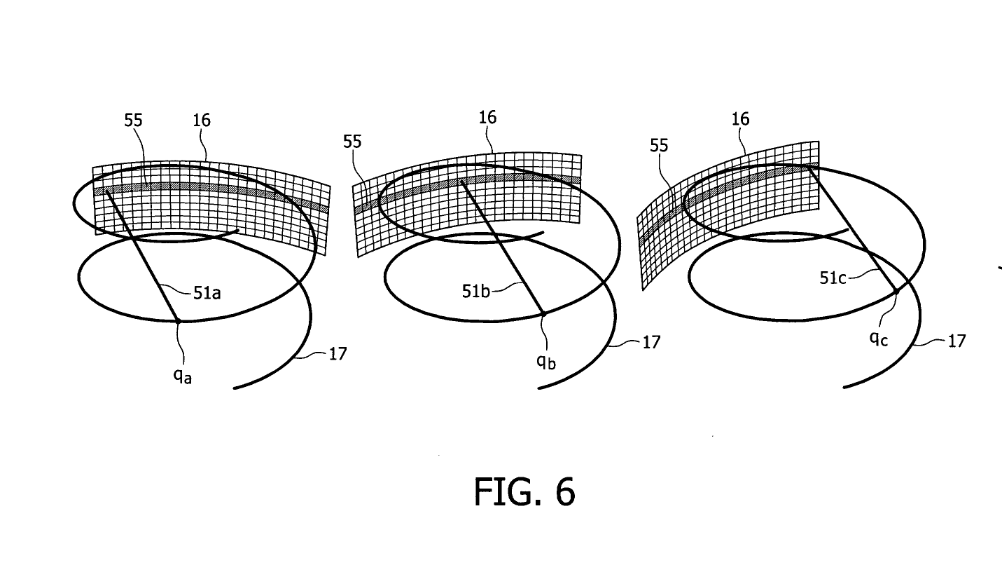

FIG. 6 A plurality of perspective views of a helical section, from which parallel beams originate from different radiation source positions,

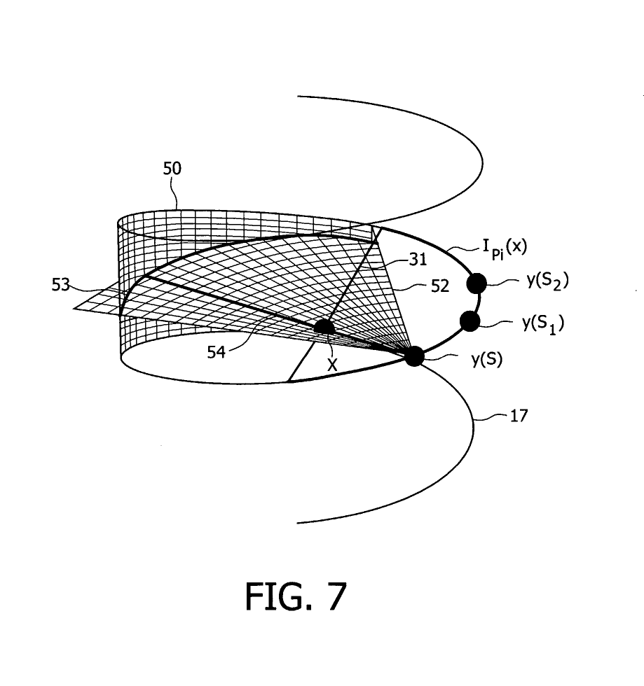

FIG. 7 Schematic perspective view of a helical section with a compartmentalized section of a κ-plane and κ-line,

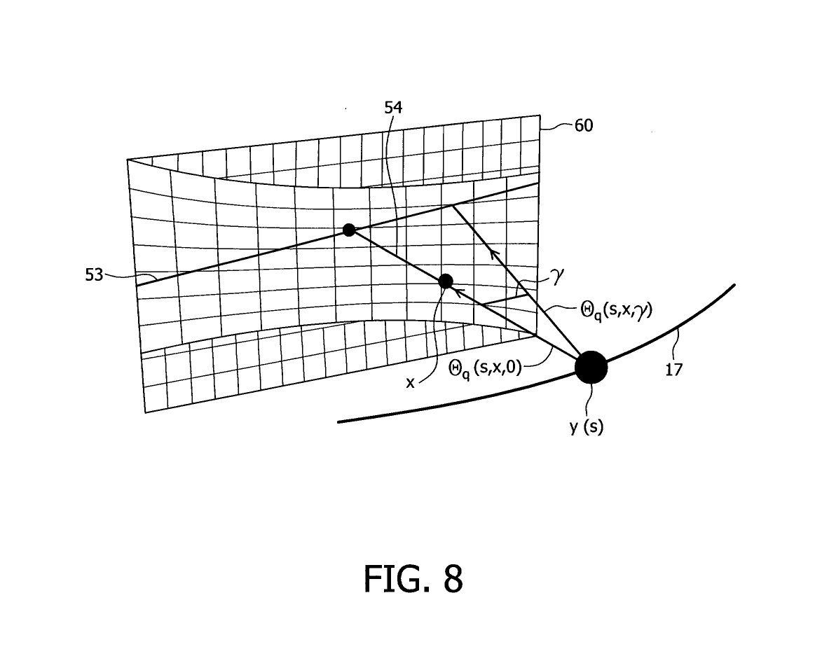

FIG. 8 A schematic perspective view of a helical section, a κ-angle γ and a κ-line on a virtual planar detector surface,

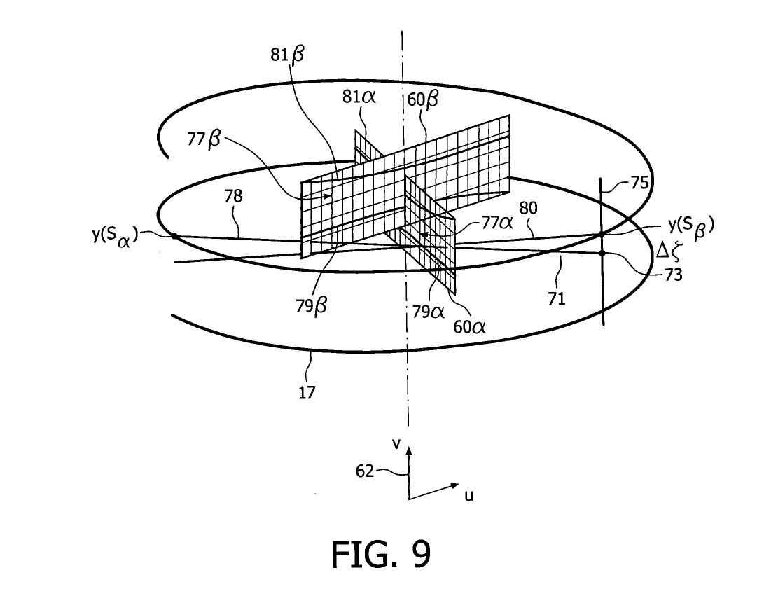

FIG. 9 to FIG. 11 A helical section, two positions of the virtual planar detector surface for two radiation source positions, a fictitious beam and a real beam and

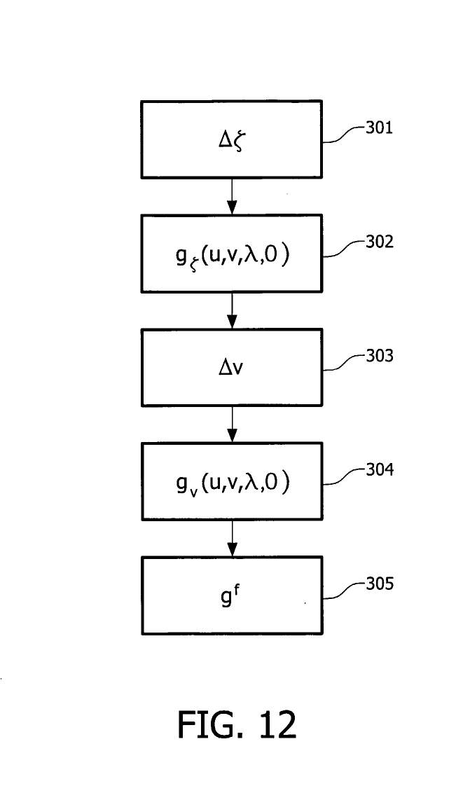

FIG. 12 A flow chart for determination of the fictitious measured value, according to the invention.

The computer tomograph shown in FIG. 1 comprises a gantry 1, which can rotate about an axis of rotation 14 that runs parallel to the z direction of the coordinate system 22 shown in FIG. 1. For this purpose, the gantry 1 is driven by a motor 2 with a preferably constant but adjustable angular velocity. A radiation source S is fastened to the gantry 1, e.g. an X-beam emitter. This is equipped with a collimator arrangement 3, which fades out a bundle of rays 4 from the radiation generated by the radiation source S i.e. a beam which has a finite non-zero extension in the z direction as well as in a direction perpendicular to it (i.e. in a plane perpendicular to the axis of rotation).

The bundle of rays 4 passes through a cylindrical examination area 13 in which an object, for example a patient can be present on a patient positioning table (neither are shown) or also a technical object can be located. After the passing through of the examination area 13, the bundle of rays 4 strikes a detector unit 16 fastened to the gantry 1, which detector unit comprising a multiplicity of detector elements, which are arranged in the form of a matrix in rows and columns in this embodiment. The detector columns run parallel to the axis of rotation 14. The detector rows are arranged in planes perpendicular to the axis of rotation, in this embodiment on a circular arc around the radiation source S (focus-centered detector surface). In other embodiments, they could, however, also be formed in a different way, e. g. they can describe a circular arc about the described axis of rotation 14 or be a straight line. Each of the detector elements hit by the bundle of rays 4 delivers a measured value for a beam from the bundle of rays 4 in every radiation source position.

The angle of beam spread, if the bundle of rays 4, described with aₘₐₓ defines the diameter of the object cylinder, within which the object to be examined is located when the measured values are acquired. The angle of beam spread is defined as the angle enclosed by a beam at the edge of the bundle of rays 4 in a plane perpendicular to the axis of rotation 14 and a plane defined by the radiation source S and the axis of rotation 14.

The examination area 13 or the object or the patient table can be displaced by means of a motor 5 parallel to the axis of rotation 14 or displaced with respect to the z axis. But equivalent to this the gantry could also be displaced in this direction. If this is a technical object and not a patient, the object can be rotated in the course of the examinationinvestigation, while the radiation source S and the detector unit 16 are at rest.

If the motors 2 and 5 run simultaneously, the radiation source S and the detector unit 16 describe a helical trajectory 17 relative to the examination area 13. If, on the other hand, the motor 5 for advancing in the direction of the axis of rotation 14 stands still and the motor 2 allows the gantry to rotate, there is a circular trajectory for the radiation source S and the detector unit 16 relative to the examination area 13. Only the helical trajectory 17 is observed below.

The measured values acquired by the detector unit 16 are fed to a computing unit 10, which is connected to the detector unit 16 e. g. through a data transmission unit (not shown) working by a contactless method. The computing unit 10 calculates fictitious measured values, reconstructs the absorption distribution in the examination area 13 from the fictitious and real measured values and reproduces them, for example on a monitor 11. The two motors 2 and 5, the computing unit 10, the radiation source S and the transfer of the measured values from the detector unit 16 to the computing unit 10 are controlled by a control unit 7.

The individual steps of an embodiment of the computed tomography method as invented are elucidated below with the help of the flow chart in FIG. 2.

After the initialization in step 101 the gantry rotates at an angular velocity, which is constant in this example of embodiment. But it can also vary, e.g. in relation to time or the radiation source position.

In step 102 the examination area or the patient positioning table is moved parallel to the axis of rotation 14 and the radiation of the radiation source S is switched on, so that the detector unit 16 can detect the radiation from numerous angular positions s and the radiation source S moves relative to the examination area 13 on the helical trajectory 17. In this manner, the real measured values are acquired.

A provisional object image is first reconstructed from the acquired real measured values in step 103. This reconstruction is effected here by a known exact method, which is described for example in “Analysis of an Exact Inversion Algorithm for Spiral Cone-Beam CT”, Physics Medicine and Biology, vol. 47, pp. 2583-2597 (further denoted by E2) and is designated hereafter as κ-method. The individual steps of the κ-method are described below in combination with the flow chart in FIG. 3, where the following equation from E2 is cited for understanding this flow chart:

$$f ( x ) = - \frac{1}{2 {π}^{2}} {\int }_{{I}_{Pi} ( x )} ⅆ s \frac{1}{\vert x - y ( s ) \vert} {\int }_{- π}^{π} \frac{ⅆ γ}{ⅆ sin γ} \frac{\partial }{\partial q} {D}_{f} ( y ( q ) , Θ ( s , x , γ ) ) {\vert}_{q = s} . \qquad ( 1 )$$

This equation describes an exact reconstruction of the absorption through back projection of the measured values. Here f(x) denotes the spatial absorption distribution in the examination area at the point x and I_{Pi}(x) denotes the part of the helix which is enclosed by a Pi-line 31.

The Pi line 31 of an object point 35 at the point x in the examination area and I_{Pi}(x) are shown in FIG. 4 and FIG. 5. The radiation source S moves relatively to the examination area around an object point 35 on the helical path 17. The Pi-line 31 is here the straight line that intersects the helix 17 at two points and the object point 35, where the helical sector I_{Pi}(x) enclosed by the line subtends an angle smaller than 2π.

Furthermore, in equation (1), s is the angular position of the radiation source S on the helix 17 relating to an arbitrary but fixed reference angle position and y(s) is the position of the radiation source S in three dimensional space, which is parameterized by the following equation:

$$y ( s ) = ( \begin{matrix}R cos s \\ R sin s \\ \frac{s}{2 π} h\end{matrix} ) \qquad ( 2 )$$

Here R is the radius of helix 17 and h is the pitch, i.e. the distance between two positions on the helix 17, which have a mutual angular distance on the helix of Δs=2π.

The expression D_{f}(y,Θ) describes the measured value, which is assigned to a beam that emerges from the radiation source position y(s) and runs in the direction of the unit vector Θ(s,x,γ). The measured value D_{f}(y,Θ) can be expressed by the following line integral:

$${D}_{f} ( y ( q ) , Θ ) = {\int }_{0}^{∞} ⅆ l f ( y + l Θ ) . \qquad ( 3 )$$

According to the equation (1) the real measured values are derived in step 201 first partially according to q, i.e. the angular position of the radiation source. It should be remembered that only y depends on q, but not Θ, so that measured values of parallel beams are to be taken into consideration for the derivation.

As parallel beams have the same cone angle (angle subtended by this beam with a plane oriented perpendicularly to the axis of rotation 14 and containing the radiation source position, from which this beam emerges), parallel beam s 51a, 51b, 51c meet on the same detector line 55 for the focus-centered detector surface 16 (see FIG. 6, in which only a partial area of the detector surface 16 is shown). For partial derivation, the measured values can therefore be re-sorted first. In addition, measured values pertaining to parallel beams 51, thus to the same detector line 53, but to different angular positions qₐ, q_{b}, q_{c} of the radiation source are collated to one quantity each. The measured values of each quantity are then derived e.g. numerically by the known finite element method according to the angle position q of the radiation source, where known smoothening techniques can be used.

The unit vector Θ depends on the κ-angle γ, which can be described with the help of what are called κ-planes 52. The κ-planes and the κ-angle γ are elucidated below.

For determining a κ-plane 52, a function

$${s}_{1} ( s , {s}_{2} ) = \{ \begin{matrix}\frac{m {s}_{2} + ( n - m ) s}{n} , s \leq {s}_{2} < 2 + 2 π \\ \frac{m s + ( n - m ) {s}_{2}}{n} , s > {s}_{2} > s - 2 π\end{matrix} \qquad ( 4 )$$

is introduced, which depends on non-negative, integer values n and m, n>m. In this example of embodiment, selected values are n=2 and m=1. But also other values can be selected for n, m. The equation (1) would still remain exact, only the position of the κ-planes 52 would change.

For determining the κ-plane 52 for a measured value, whose assigned beam emerges from the radiation source position y(s) and runs through the position x in the examination area, a value s₂ ε I_{Pi}(x) is selected such that y(s), y(s₁(s,s₂)), y(s₂) and x are in one plane. This plane is designated as κ-plane 52 and the interface 53 between the κ-plane 52 and the detector surface 50 as κ-line 53. FIG. 7 shows a fan-like section of a κ-plane 52.

The detector surface 50 in FIG. 7 is a fictitious detector surface 50 which runs on a fictitious cylinder surface defined by the path of the helix 17. The κ-line 53 runs accordingly on this detector surface 50. In FIG. 8 the κ-line 53 runs on a fictitious planar detector surface 60, which will be further elucidated below. The exact reconstruction and especially the filtering described in step 202 can be carried out on any arbitrary detector surface if the measured values are projected on the respective detector surface and the respective intersecting line of the κ-plane is determined with the respective detector surface.

The vector Θ(s,x,γ) shows for γ=0 in the direction of the beam 54, which emerges from y(s) and passes through the point x in the examination area (see FIG. 8). For γ≠0

a) Θ(s,x,γ) lies in the κ-plane 52, which has been determined for the measured value, whose assigned beam emerges from y(s) and passes through the point x in the examination area,

b) Θ(s,x,γ) emerges from the radiation source position y(s) and

c) encloses the angle γ with the vector Θ(s,x,0), where, for two vectors Θ(s,x,γ₁) and Θ(s,x,γ₂), their κ-angles γ₁, γ₂ have different signs, the vector Θ(s,x,0) lies between these two vectors Θ(s,x,γ₁) and Θ(s,x,γ₂). The κ-angle γ thus has a sign.

The κ-angle γ describes, as an integration variable in equation (1), the sampling of the derived measured values ∂D_{f}(y(q), Θ(s,x,γ))/∂q along the κ-line 53. The integration will be dealt with in detail below in step 202 in combination with the filtering of the derived measured values.

Reference is made to E2 for a more detailed explanation of the κ-planes, κ-lines and κ-angle.

In step 202, the measured values derived in step 201 are filtered i.e. the integration is executed via the κ-angle γ according to equation (1).

In addition, for each beam, whose assigned measured value is taken into consideration in the reconstruction i.e. for each combination of a point x in the examination area that has been passed through by a beam, and an angular position s ε I_{Pi}(x) a \tilde{κ} line is determined as filter line, while, as explained above, a value s₂ ε I_{Pi}(x) is selected such that y(s), y(s₁(s,s₂)), y(s₂) and x lie in one plane, the \tilde{κ} plane. The \tilde{κ} line is then determined as line of intersection between the \tilde{κ} plane and the respective detector surface. In this example of embodiment is determined the line of intersection concerned on the focus-centered detector surface 18.

For filtering a measured value, which is taken into consideration for the reconstruction, first the filter line pertaining to this measured value i.e. the \tilde{κ} line is determined. Along this filter line, measured values lying on the filter line are each multiplied by a κ-factor in the filter direction and summed. The κ-factor decreases with increase in the sine of the \tilde{κ} angle γ. It is particularly equal to the reciprocal of the sine of the \tilde{κ} angle γ. The result of the summation is the filtered measured value. This is repeated for all measured values which are to be taken into consideration for the reconstruction.

In the following steps, the filtered measured values are back-projected for the reconstruction of the absorption distribution in the examination area 13 in essence according to the integration through s in equation (1).

In step 203 a location x and a Voxel V(x) arranged at this location is specified within a specifiable area (field of view—FOV) in the examination area 13, which has not yet been reconstructed in the back-projection steps that may already have preceded.

Then, in step 204 the quantity of angular positions s ε I_{Pi}(x) or radiation source positions y(s) is determined with s ε I_{Pi}(x), from which beams emerge, which pass through the Voxel V(x) in the center.

Then in step 205, an angular position s from the quantity determined in step 204 is predefined for angular positions, which angular position has not yet been used for reconstruction of the voxel V(x).

In step 206, a measured value is determined for the beam emerging from the radiation source position y(s) defined by the predefined angular position s and passing through the voxel V(x) centrally. If the detector surface 18, as in this example of embodiment, is composed of a plurality of rectangular detector elements each recording a measured value, and if the beam strikes a detector element centrally, the measured value picked up by this detector element is determined for this beam. If this beam does not strike a detector element centrally, then a measured value is determined by interpolation of the measured value recorded by the detector element which the beam hits, and neighboring measured values, for example by a bilinear interpolation.

The measured value determined in step 206 is multiplied in step 207 by a weight factor, which becomes progressively smaller with increasing distance of the radiation source y(s) from the location x specified in step 201. In this embodiment, this weight factor, according to equation (1), is equal to 1/|x−y(s)|.

In step 208 the weighted measured value is added to the voxel V(x), which is initially equal to zero in this example of embodiment.

In step 209 it is checked whether all angular positions s from the number of angular positions determined in step 204 have been taken into consideration for the reconstruction of the voxel V(x). If this is not the case, then the flow chart branches at step 205. Otherwise it is checked in step 210 whether all voxels V(x) have been reconstructed in FOV. If not, step 203 is proceeded with. If, on the contrary, all voxels V(x) in FOV have been run through, the absorption has been determined in the entire FOV and the provisional object image has been reconstructed.

After the provisional object image has been reconstructed in steps 201 to 210 or in step 103 respectively, a fictitious beam is specified in step 104, for which beam a fictitious measured value is to be determined. Furthermore, in step 104 a real beam is predefined whose assigned real measured value mainly contributes to the determination of the fictitious measured value assigned to the fictitious beam, as described below. Basically, any beam that passes through the object can be selected as a fictitious beam, according to the invention. The two beam s are so selected that the real and the fictitious beams lie on the same straight line, as observed in a direction oriented parallel to the axis of rotation 14. This means that the fictitious radiation source position from which the fictitious beam emerges and the real radiation source position from which the real beam emerges are selected such that they lie on one straight line parallel to the axis of rotation. Furthermore, the meeting points of the real and the fictitious beams are selected such that they lie on the real detector surface on a straight line parallel to the axis of rotation i.e. they are selected such that they meet on the detector surface in the same detector column.

The selection of the fictitious beam 71 and of the real beam 80, as they are shown in this example of embodiment, is described in detail with the help of FIG. 9.

FIG. 9 shows the helix 17, on which the radiation source moves relative to the examination area 13. Moreover, a virtual planar detector surface 60α, 60β is shown in two positions. The virtual, planar detector surface rotates, as the real focus-centered detector surface 18, with the radiation source. In FIG. 9, the position of the virtual detector surface is denoted as 60α, if the radiation source is at the position y(s_{α}), and the position of the virtual detector surface as 60β, if the radiation source is at the position y(s_{β}). The virtual detector surface is oriented such that the normal to the detector surface at the center of the virtual detector surface runs through the respective radiation source position. Furthermore, the virtual detector surface is oriented such that it contains the axis of rotation 14.

As already explained above, those measured values are exclusively used whose beams emerge from the respective helical section I_{Pi}(x) that is bounded by the respective Pi-line 31, during the reconstruction by means of a \tilde{κ} method for reconstruction of a Voxel V(x) at the position x in the examination area 13. This condition means obviously the same as the condition that a beam or the corresponding measured value should be taken into consideration only for reconstruction of the Voxel V(x) at the position x, only if it meets the detector surface 60α, 60β within what is called the Pi-window 77α, 77β. The Pi-window 77α, 77β is bounded on the virtual planar detector surface 60α, 60β by two Pi-boundary lines 79α, 81α or 79β, 81β, whose path is indicated below.

First, a direct beam 78 is selected, which, starting from a real radiation source position y(s_{α}) passes through the virtual planar detector surface 60α in the Pi-window 77α. The fictitious beam 71 is the beam that is oriented exactly opposed to the direct beam 78. The virtual radiation source position 73, from which the fictitious beam 71 starts, is the intersection point 73 of the fictitious beam 71 with an intersecting straight line 75, which is oriented such that it cuts the helix 17 and the fictitious beam 71.

The real beam 80 to be selected starts from the real radiation source position y(s_{β}), which is closest to the fictitious radiation source position 73 on the intersecting straight line 75. Furthermore, the real beam 80 is oriented such that the real beam 80 and the fictitious beam 71 lie on a straight line, as observed in a direction oriented parallel to the axis of rotation 14, i.e. the two beams 73, 80 pass through a plane in which the virtual planar detector surfaces 60α, 60β lie, at positions which lie on a straight line parallel to the axis of rotation 14.

In another embodiment as invented a real beam can be determined from these real beams if a plurality of real beams are oriented such that they lie on a straight line common with the fictitious beam 71, if observed in a direction parallel to the axis of rotation 14 that has the shortest distance from the fictitious beam 71.

In step 105 the acquired measured values, whose assigned beams start from the real radiation source position y(s_{β}) determined in step 104, are projected on the virtual planar detector surface 60β along these beams.

A position on the virtual detector surface 60α, 60β can be described by the coordinates (u,v), where u and v are coordinates of a Cartesian coordinate system on the planar detector surface 60α, 60β. The u-coordinates axis is then vertical and the v-coordinate axis has an orientation parallel to the axis of rotation 14. This coordinates system 62 is shown in the FIG. 9 below the planar detector surface, for the sake of clarity. The origin of the coordinate system however lies at the center of the detector surface, i.e. where the normal of the planar detector surface 60β running through the radiation source position y(s_{β}), stands on the planar detector surface 60β.

FIG. 9 shows the coordinate system 62 for the position 60β of the virtual planar detector surface. For the position 60α of the virtual planar detector surface the coordinate system 62 is to be rotated accordingly.

The relation of the coordinates on the fictitious planar detector surface 60α, 60β to the directions of radiation is given by the following equations:

$$\begin{matrix}u = R tan β and & ( 5 ) \\ v = \sqrt{{R}^{2} + {u}^{2}} tan λ = R \frac{tan λ}{cos β} . & ( 6 )\end{matrix}$$

Here, λ is the cone angle of a beam. Furthermore, β is the fan angle of a beam i.e. the angle enclosed by this beam with a plane containing the axis of rotation 14 and the radiation source position.

The path of the Pi-boundary lines 79α, 81α or 79β, 81β on the fictitious planar detector surface 60α or 60β as the case may be, can be described by the following equations:

$$v ( u ) = + \frac{h}{2 π} ( 1 + {( \frac{u}{R} )}^{2} ) ( \frac{π}{2} - arctan \frac{u}{R} )$$

$$and$$

$$v ( u ) = - \frac{h}{2 π} ( 1 + {( \frac{u}{R} )}^{2} ) ( \frac{π}{2} + arctan \frac{u}{R} ) .$$

The Pi-boundary lines 79α, 81α or 79β, 81β can be projected on the detector surface 18 of the detector unit 16 along the real beams which start from the respective real radiation source position y(s_{α}) or y(s_{β}) respectively. Reconstruction methods, which use exclusively those measured values that lie on the virtual planar detector surface 60α, 60β or on the detector surface 18 of the detector unit 16 between the Pi-boundary lines, are designated as Pi-reconstruction methods.

In step 106 the fictitious measured value g^{f} is calculated with the help of a Taylor development. The Taylor development gives the following equation in the first approximation
g^{f}=g(u_{β},v_{β},s_{β},0)+Δζ·g_{ζ}(u_{β},v_{β},s_{β},0)+Δv·gᵥ(u_{β},v_{β},s_{β},0) (7)

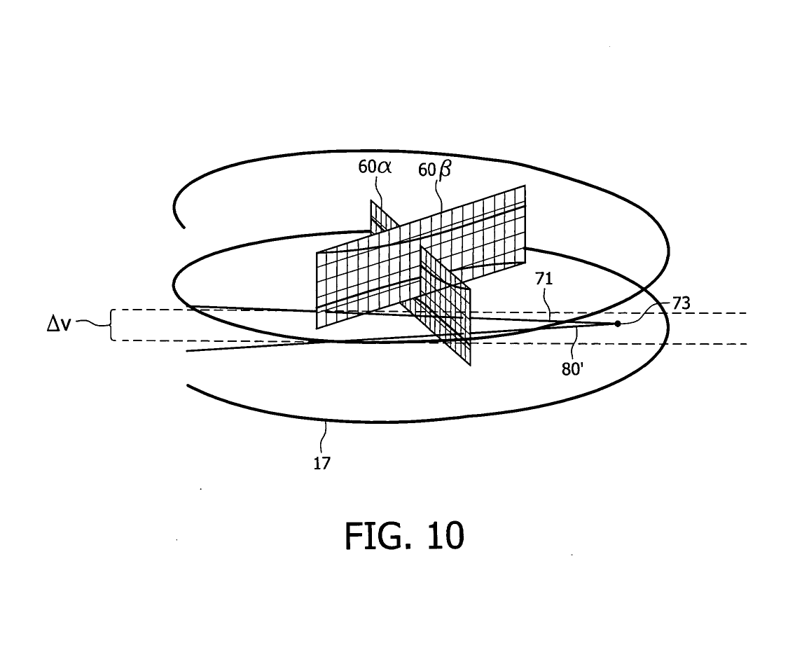

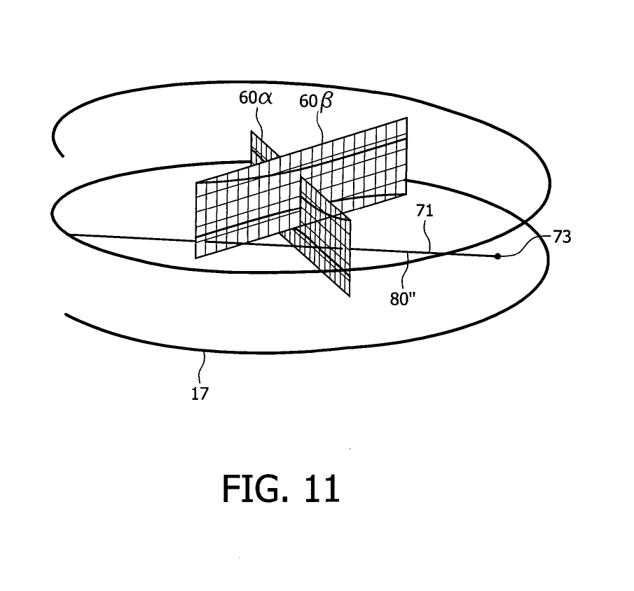

This Taylor development can be interpreted on the fictitious beam 71 as a displacement of the real beam 80, whose measured value is described by g(u_{β},v_{β},s_{β},0). This means that the real beam 80 is first displaced by Δζ parallel to the axis of rotation 14 in such a manner that the real radiation source position lies on the fictitious radiation source position 73. The displaced real beam is denoted as 80′ in FIG. 10. The passing point of the displaced real beam 80′, on which the displaced real beam 80′ passes through the plane in which the virtual planar detector surface 60α lies, is then displaced by the distance Δv parallel to the axis of rotation 14 in such a manner that it coincides with the point of incidence of the fictitious beam 71 on the virtual, planar detector surface 60β. The real beam finally displaced in this manner is designated as 80″ in FIG. 11 and lies on the fictitious beam 71.

In equation (7), g^{f} is the fictitious measured value to which the fictitious beam 71 specified in step 104 is assigned.

The real measured value g(u_{β},v_{β},s_{β},0) is the real measured value that has been generated by the real beam 80 determined in step 104, which beam meets the virtual planar detector surface 60β from the real radiation source position y(s_{β}) and at the location (u_{β},v_{β}).

The individual steps for determining the fictitious measured value according to the equation (7) are explained below with the help of the flow chart shown in FIG. 12.

In step 301 the distance Δζ is determined i.e. the distance between the real radiation source position y(s_{β}) and the fictitious radiation source position 73 on the intersecting straight line 75.

In step 302 the partial derivation of the real measured values is calculated according to the respective radiation source position ζ on the intersecting straight line 75 at the location (u_{β},v_{β},s_{β},0). This partial derivation is designated with g_{ζ}(u_{β},v_{β},s_{β},0). What is problematic here is that though there is a real radiation source position y(s_{β}) on the intersecting straight line 75 at the point of intersection with the helix 17, other real radiation source positions are not available on the intersecting straight line 75, however. The partial derivation g_{ζ}(u,v,s,0) is therefore calculated, as invented, not through direct derivation of the real measured values, but through partial derivation of the provisional object image reconstructed in step 103 in z direction of the coordinates system 22 shown in FIG. 1, i.e. in a direction oriented parallel to the axis of rotation 14 and by forward projection along the undisplaced real beam 80 through the partially derived provisional object image. This can be described by the following equation:

$${g}_{ς} ( u , v , s , 0 ) = {\int }_{L ( x )} {f}_{z} ( x ) ⅆ x . \qquad ( 8 )$$

Here, L(x) is the path of the real beam 80 determined in step 104 i.e. equation (3) is a line integral of the object values partially derived for z (e.g. absorption values) f_{z}(x) along the real beam 80. The partial derivation for z relates, as already mentioned above, to the coordinate system 22 shown in FIG. 1 This is thus a partial derivation in a direction oriented parallel to the axis of rotation 14.

Thus, for calculating g_{ζ}(u,v,s,0), the provisional object image reconstructed in step 103 is partially derived for z. Then the integration is done as given in equation (8), by carrying out a known forward projection through the partially derived object image along the real beam (80). The value generated hereby is the partial derivation g_{ζ}(u,v,s,0). The forward projection can be carried out in a simple manner e.g. by adding up all values f_{z}(x) lying on the real beam L(x).

In step 303, the distance Δv is determined i.e. the distance between the position at which the real beam 80′ displaced parallel to the axis of rotation 14 by Δζ passes through the plane in which the planar detector surface 60β lies and the point of incidence of the fictitious beam 71 on the planar detector surface 60β.

In step 304 the acquired real measured values g(u_{β},v_{β},s_{β},0) are partially derived on the virtual, planar detector surface 60 for the variable v at the position (u_{β},v_{β},s_{β},0). The partial derivation for v at the position (u_{β},v_{β},s_{β},0) is designated as gᵥ(u_{β},v_{β},s_{β},0). The partial derivation can be executed for example by the finite differences method.

In step 305, the fictitious measured value g^{f} is calculated according to the equation (7) by forming a first product by multiplying the distance Δζ determined in step 301 by the derivation g_{ζ}(u_{β},v_{β},s_{β},0) determined in step 303, by forming a second product by multiplying the distance Δv determined in step 303 by the derivation gᵥ(u_{β},v_{β},s_{β},0) determined in step 304 at the position (u_{β},v_{β},s_{β},0) and by adding up the first and second products.

Steps 104 to 106 and 301 to 305 can be repeated for a desired quantity of fictitious beams and fictitious measured values.

After one or more fictitious measured values have been generated from the real measured values, a final object image is reconstructed by back-projection of the real and fictitious measured values in step 107. The κ method described above in the context with steps 201 to 211 is used here, where now also fictitious measured values are taken into consideration besides the real measured values, which lie between the Pi-boundary lines. This raises the signal-to-noise ratio.

The fictitious measured values determined in step 106 are taken into consideration in this example of embodiment, by reviewing in step 208, if a fictitious measured value has been calculated in step 106, whose assigned fictitious beam 71 runs opposed to the real beam 78 determined earlier in step 206. If so, this fictitious measured values is multiplied by a weight factor that is equal to the weight factor that has been determined earlier in step 207 for the real beam i.e. for the real measured value. A mean value is formed via the weighted fictitious and weighted real measured values (e.g. the arithmetical mean value) and in step 208 the mean value is added to the respective Voxel V(x).

After the final object image has been reconstructed in step 107 the method as invented is terminated in step 108.

In other embodiments, another Pi reconstruction method can be used for example for reconstruction of the provisional and/or final object image. Preferably, according to the invention, fictitious measured values are determined whose assigned fictitious beams meet the detector surface between Pi-boundary lines where a real beam is determined for the respective fictitious beam, which real beam does not hit the detector surface between the Pi-boundary lines. The reconstruction of the final object image from the real and fictitious measured values then leads to a final object image with an improved signal-to-noise ratio.

The computed tomography method as invented is not restricted to the reconstruction method mentioned in the example of embodiment. Every reconstruction method that can reconstruct a provisional object image from the acquired real measured values and a final object image from the real and fictitious measured values can be used within the framework of the invention.

LIST OF REFERENCE DRAWINGS

| αₘₐₓ | Angle of beam spread |
|---|---|
| Δζ | Distance by which the real beam is first |
| | displaced to the axis of rotation |
| Δν | Distance, by which a penetration point of |
| | the displaced real beam is displaced |
| | parallel to the axis of rotation |
| χ | κ-angle |
| Θ_{q}(s, x, 0), Θ_{q}(s, x, γ) | Unit vectors |
| qₐ, q_{b}, q_{c} | Angular position of the radiation source |
| x | Position in the examination area |
| y(s_{α}), y(s_{β}) | Radiation source position |
| y(s), y(s₁), y(s₂) | Radiation source position |
| I_{Pi}(x) | Helical section enclosed by the Pi-line |
| S | Radiation source |
| 1 | Gantry |
| 2, 5 | Engine |
| 3 | Collimator arrangement |
| 4 | Bundle of rays |
| 7 | Control unit |
| 10 | Computing unit |
| 11 | Monitor |
| 13 | Examination area |
| 14 | Axis of rotation |
| 16 | Detector unit |
| 17 | Helical trajectory |
| 18 | Detector surface |
| 22 | Coordinate system |
| 31 | Pi-Line |
| 35 | Object point |
| 50 | Detector surface |
| 51a, 51b, 51c | Parallel beams |
| 52 | κ- Plane |
| 53 | κ- Line |
| 54 | Beam for γ = 0 |
| 55 | Detector line |
| 60 | Fictitious, planar detector surface |
| 60α, 60β | Two positions of the virtual planar |
| | detector surface |
| 71 | Fictitious beam |
| 73 | Virtual radiation source position |
| 75 | Intersecting straight line |
| 77α, 77β | Pi-window |
| 78 | Direct beam |
| 79α, 81α, 79β, 81β | Pi-boundary lines |
| 80 | Real beam |
| 80′ | Real beam displaced by Δζ |
| 80″ | Real beam displaced by Δζ and Δν |

1. Computed tomography method comprising the following steps:
a) Determination of non-acquired, fictitious measured values from real measured values, where a fictitious beam is assigned to each fictitious measured value and where the determination of a fictitious measured value has the following steps:
i) Reconstruction of a provisional object image from the real measured values,
ii) Calculation of a partial derivation of the provisional object image in a direction parallel to the axis of rotation,
b) Reconstruction of a final object image from the real measured values and the fictitious measured values.

2. Method as claimed in claim 1, comprising the following steps:
i) Determination of a real beam, which is so oriented that the real beam and the fictitious beam, which is assigned to the fictitious measured value to be determined, lie on a common straight line as viewed in the direction parallel to the axis of rotational,
ii) Determination of a partial derivation of the real measured values in the direction parallel to the axis of rotation at the position of the real measured value that is assigned to the real beam determined in step i), by forward projection along the real beam determined in step i), by the partially derived provisional object image,
iii) Determination of the fictitious measured value from the real measured values with the help of the partial derivation determined in step ii).

3. Method as claimed in claim 1, wherein the provisional object image and/or the final object image are reconstructed exactly.

4. Method as claimed in claim 1, wherein the provisional object image is reconstructed with a lower resolution than the final object image.

5. Method as claimed in claim 2, wherein if a plurality of real beams, viewed in the direction oriented parallel to the axis of rotation, the fictitious beam which is assigned to the fictitious measured value to be determined lie on a common straight line, and the real beam from these real beams that has the shortest distance to the fictitious beam is determined in step i).

6. Method as claimed in claim 2, wherein the determination of the real beam has the following steps in step i):
α) Determination of a intersecting straight line, which runs parallel to the axis of rotation and cuts the fictitious beam, which is assigned to the fictitious measured values to be determined, and also cuts the helix,
β) Determination of an intersection point of the intersecting straight line with the fictitious beam as a fictitious radiation source position, from which the fictitious beam emerges,
γ) Determination of those real radiation source positions on the helix that lies on the intersecting straight line and is closest to the fictitious radiation source position and
δ) Determination of the real beam emerging from the radiation source position determined in step γ), which real beam is so oriented that the fictitious beam and the real beam viewed in a direction oriented parallel to the axis of rotation lie on a common straight line.

7. Method as claimed in claim 2, wherein the determination of the fictitious measured value has the following steps in step iii):
A) Projection of the real measured values, whose assigned beams start from the real radiation source position determined in step γ), along these beams on a virtual planar detector surface, which contains the axis of rotation and whose central surface normal passes through the real radiation source position determined in step γ),
B) Determining the distance between the fictitious radiation source position and the real radiation source position determined in step γ),
C) Determining the distance of the points of incidence of the fictitious beam and a beam hat is displaced by the distance determined in step B) relative to the real beam parallel to the axis of rotation on a plane that contains the virtual, planar detector surface,
D) Formation of the partial derivation of the real measured values, whose assigned beams start from the real radiation source position determined in step γ), for the position of the respective real measured value on a straight line oriented parallel to the axis of rotation at the position of the real measured value, which is assigned to the real beam determined in step δ) and
E) Determining the fictitious measured value by forming a first product by multiplication of the distance determined in step B) by the partial derivation determined in step ii) by forming a second product by multiplication of the distance by the partial derivation and by summing the first and second products.

8. Computed tomography method as claimed in claim 7, wherein the detector unit has a detector area and that fictitious measured values are determined in step D) whose assigned fictitious beams strike the detector surface between Pi-boundary lines, that in step i) for the fictitious measured value to be determined, a real beam is determined, that does not strike the detector surface between the Pi-boundary lines and that the final object image is reconstructed with a Pi-reconstructive method.

9. Computer tomograph comprising
a detector unit coupled to a radiation source for acquisition of real measured values,
a computing unit for determining fictitious values from real measured values and for reconstructing an object image from the real measured values and the fictitious values.

10. Computer program for a control unit for controlling a radiation source, a detector unit, a drive arrangement and a computing unit of a computer tomograph according to the steps as claimed in claim 1.
